(19) United States
(12) Patent Application Publication
(10) Pub. No.: US 2025/0366820 A1
(43) Pub. Date: Dec. 4, 2025

(54) ULTRASONIC DIAGNOSTIC APPRATUS, ULTRASONIC DIAGNOSTIC PROGRAM, AND ULTRASONIC DIAGNOSTIC METHOD

(71) Applicant: KONICA MINOLTA, INC., Tokyo (JP)

(72) Inventor: Yoshihiro TAKEDA, Tokyo (JP)

(21) Appl. No.: 19/222,219

(22) Filed: May 29, 2025

(30) Foreign Application Priority Data
May 31, 2024 (JP) 2024-088953

(51) Int. Cl. A61B 8/08; A61B 8/00; G06T 7/73; G06T 11/00; G16H 50/20

(52) U.S. Cl. CPC A61B 8/0891; A61B 8/461; A61B 8/469; A61B 8/488; A61B 8/5223; G06T 7/73; G06T 11/001; G16H 50/20; G06T 2207/10132; G06T 2207/30101; G06T 2210/41; G06T 2210/62

(57) ABSTRACT

Provided is an ultrasound diagnostic apparatus that includes a display on which information indicating a position of a blood vessel region recognized using a learned model is displayed; and an operation unit which is adapted to change a degree of transparency of the information indicating the position of the blood vessel region.

### CROSS REFERENCE TO THE RELATED APPLICATION

The entire disclosure of Japanese patent Application No. 2024-088953, filed on May 31, 2024, is incorporated herein by reference in its entirety.

### BACKGROUND

### 1. Technological Field

The present invention relates to an ultrasonic diagnostic apparatus, an ultrasonic diagnostic program, and an ultrasonic diagnostic method.

### 2. Description of the Related Art

Conventionally, many medical diagnoses based on ultrasonic images of organs and tissues of living tissues have been performed. An ultrasonic diagnostic apparatus that generates an ultrasonic image transmits an ultrasonic wave to a living body, receives an echo reflected by an organ or a tissue, and images the echo as a cross-sectional image. In addition, in recent years, a technique has been known in which a blood vessel region represented in an ultrasonic image is displayed as an annotation by using artificial intelligence.

For example, as disclosed in Japanese Unexamined Patent Publication No. 2023-026610, a technology is known in which an ultrasonic image generated by an ultrasonic diagnostic apparatus is analyzed, and a recognized blood vessel region is highlighted and displayed. Specifically, in Patent Document 1, typical pattern data of a blood vessel region is stored in advance as a template, and the degree of similarity with respect to the pattern data is calculated while searching the ultrasound image with the template. Then, a place where the degree of similarity is equal to or more than a threshold value and is maximum is regarded as a place where a blood vessel region exists. Furthermore, for the calculation of the degree of similarity, a general image recognition method or the like using machine learning or deep learning can be used in addition to template matching.

In this specification, techniques such as machine learning and deep learning for recognizing a blood vessel region are collectively referred to as artificial intelligence (AI).

### SUMMARY

According to the above-described related art, since it becomes easy to grasp a blood vessel region, it is useful when performing a procedure of inserting an insertion object such as a puncture needle and a catheter into a blood vessel of a subject, so-called puncture. However, there are also cases where the artificial intelligence incorrectly recognizes the blood vessel region. Therefore, when an annotation is displayed by superimposing a predetermined color on a blood vessel detected by artificial intelligence, it may be difficult to visually recognize a tissue other than a blood vessel region located in the vicinity of the annotated blood vessel region due to erroneous recognition, or a structure having a similar shape on an image, such as a nerve, may be displayed as an annotation. Therefore, for example, when a health care professional punctures a patient while viewing an ultrasonic image, there is a risk that a nerve region of the patient may also be injured.

Objects of the present invention are to provide an ultrasonic diagnostic apparatus, a non-transitory computer-readable recording medium storing an ultrasonic diagnostic program, and an ultrasonic diagnostic method that can also display a tissue other than an annotated blood vessel region with high visibility in a case where the blood vessel region is annotation-displayed by artificial intelligence.

To achieve at least one of the abovementioned objects, according to an aspect of the present invention, the followings are provided.

(1) An ultrasound diagnostic apparatus including: a display on which information indicating a position of a blood vessel region recognized using a learned model is displayed; and operation means for changing transparency of the information indicating the position of the blood vessel region.

(2) The ultrasound diagnostic apparatus according to (1), wherein the operation means is provided on a screen of the display.

(3) The ultrasound diagnostic apparatus according to (1), wherein the information indicating the position of the blood vessel region is semitransparent color information to be superimposed on the ultrasound image.

(4) The ultrasound diagnostic apparatus according to (1), wherein the degree of transparency is changeable by the operation means during real-time display of the B-mode image on which the information indicating the position of the blood vessel region is superimposed.

(5) The ultrasound diagnostic apparatus according to (1), wherein the information indicating the position of the blood vessel region is color information indicating that the blood vessel region recognized using the learned model is painted in a predetermined color.

(6) The ultrasound diagnostic apparatus according to (1), wherein the information indicating the position of the blood vessel region is color information in which a boundary region of the blood vessel region recognized using the learned model is displayed in a frame shape.

(7) The ultrasound diagnostic apparatus according to (1), wherein the display shows information indicating a position of a nerve region recognized using a learned model, and the ultrasound diagnostic apparatus further includes operation means for enabling a change in transparency of the information indicating the position of the nerve region.

(8) The ultrasound diagnostic apparatus according to (1), wherein the information indicating the position of the nerve region recognized using the learned model and the information indicating the position of the blood vessel region are simultaneously displayed on the display, and a transparency of the information indicating the position of the blood vessel region and a transparency of the information indicating the position of the nerve region are independently changeable.

(9) The ultrasonic diagnostic apparatus according to (8), wherein the display simultaneously displays the information indicating the position of the nerve region recognized using the learned model and the information indicating the position of the blood vessel region, and the ultrasonic diagnostic apparatus further includes a first operation unit capable of changing transparency of the information indicating the position of the blood vessel region and a second operation unit capable of changing transparency of the information indicating the position of the nerve region.

(10) The ultrasound diagnostic apparatus according to (1), wherein the display shows information indicating the position of the nerve region recognized using the learned model and information indicating the position of the blood vessel region at the same time, and the ultrasound diagnostic apparatus further includes one operation means capable of changing transparency of the information indicating the position of the blood vessel region and capable of changing transparency of the information indicating the position of the nerve region.

(11) The ultrasound diagnostic apparatus according to (7), wherein one of the information indicating the position of the blood vessel region and the information indicating the position of the nerve region is preferentially displayed.

(12) The ultrasound diagnostic apparatus according to (1), wherein the display is capable of simultaneously displaying information indicating the position of the muscle tissue region recognized by the learned model, information indicating the position of the nerve region recognized by the learned model, and information indicating the position of the blood vessel region.

(13) The ultrasound diagnostic apparatus according to (12), wherein a transmittance of the information indicating the position of the muscle tissue region is changeable.

(14) The ultrasound diagnostic apparatus according to (1), further including a controller that, in a case where the transparency is set to be equal to or higher than a predetermined threshold and an end instruction is provided, detects the end instruction and performs control to change the transparency to be equal to or lower than the predetermined threshold.

(15) The ultrasound diagnostic apparatus according to (1), further including a notification unit that provides a notification when the degree of transparency is set to be equal to or higher than a predetermined threshold.

(16) The ultrasound diagnostic apparatus according to (1), further including a setting unit configured to set a maximum value of a setting range of the transmittance to be equal to or less than a predetermined threshold.

(17) The ultrasound diagnostic apparatus according to (1), wherein a degree of transparency of color of the information indicating the position of the blood vessel region is set to be equal to or less than a predetermined threshold at the time of display activation for starting to display the information indicating the position of the blood vessel region.

(18) The ultrasonic diagnostic apparatus according to (1), wherein the ultrasound diagnostic apparatus is adapted to enable color Doppler display to display an overlay on the information indicating the position of the blood vessel region when a live scanning is performed in a B mode and the information indicating the position of the blood vessel region is displayed.

(19) The ultrasonic diagnostic apparatus according to (18), in which the color Doppler display can be ended while the live scan is performed in the B mode and the information indicating the position of the blood vessel region is displayed.

(20) A computer readable storage medium which stores a program implemented by a computer, the program causing the computer to execute; changing a degree of transparency of information indicating a position of a blood vessel region which is recognized using a learned model; and displaying the information indicating the position of the blood vessel region.

(21) An ultrasonic diagnostic method implemented by a computer, the method including causing the computer to execute: changing a degree of transparency of information indicating a position of a blood vessel region which is recognized using a learned model, and; displaying the information indicating the position of the blood vessel region.

### Effects of the Invention

According to the present invention, it is possible to display a tissue other than the highlighted blood vessel region with high visibility while grasping the blood vessel region by artificial intelligence.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a block diagram illustrating a configuration of an ultrasonic diagnostic apparatus of an embodiment.

FIG. 2 is a diagram illustrating a display example of a display.

FIG. 3A is a diagram illustrating a shape of information indicating a position of a blood vessel region or a neural region.

FIG. 3B is a diagram illustrating another form of information indicating the position of a blood vessel region or a neural region.

FIG. 3C is a diagram illustrating another form of information indicating the position of a blood vessel region or a neural region.

FIG. 3D is a diagram illustrating another form of information indicating the position of a blood vessel region or a neural region.

FIG. 4A is a view showing a setting button for updating the transparency configured by the spin button.

FIG. 4B is a diagram illustrating a setting button for updating the transparency, which is formed with a slider.

FIG. 5 is a flowchart for explaining an operation of superimposing and displaying information indicating the position of a blood vessel region.

FIG. 6 is a view illustrating a display on which information indicating the position of a blood vessel region and information indicating the position of a nerve region are superimposed and displayed on a B-mode image.

FIG. 7 is a flowchart for explaining an operation of simultaneously displaying information indicating the positions of a blood vessel region and a nerve region on a B-mode image in a superimposed manner.

FIG. 8 is a flowchart for explaining an operation of preferentially displaying one of information indicating the positions of a blood vessel region and a nerve region.

### DETAILED DESCRIPTION OF THE EMBODIMENTS

Hereinafter, one or more embodiments of the present invention will be described with reference to the drawings. However, the scope of the invention is not limited to the disclosed embodiments

Hereinafter, embodiments of the present invention will be described in detail with reference to the drawings. The ultrasonic diagnostic apparatus of the embodiment recognizes information indicating the position of a blood vessel region from a brightness (B) mode image of an ultrasonic echo of a subject by using a learned model of machine learning or deep learning. Next, the ultrasound diagnostic apparatus superimposes information indicating the recognized position of the blood vessel region on the B-mode image and displays the superimposed image on the display, thereby highlighting the blood vessel region. Furthermore, the ultrasound diagnostic apparatus includes operation means for changing the transparency of the information indicating the position of the blood vessel region to be superimposed and displayed on the display so as to adjust the visibility of the B-mode image behind. Accordingly, tissues other than the highlighted blood vessel region can also be displayed with high visibility. Note that hereinafter, the B-mode image may be referred to as an ultrasonic echo image.

Specifically, since the visibility of the B-mode image is improved by changing the transmittance of the information indicating the position of the blood vessel region to be higher after the position of the blood vessel region is grasped, it becomes easier to see that a minute blood vessel is injured. Furthermore, the ultrasonic diagnostic apparatus according to the present embodiment has effects such as not obstructing checking of bleeding around a blood vessel and visual recognition of intrablood vessel plaque, and suppressing overlooking of a vein that is greatly deformed by pressing of the probe. Further, a gate is set in the B mode in order to display in the Doppler mode. At that time, there is a problem that it is difficult to perform gate setting when the information indicating the position of the blood vessel region is highlighted, but since the visibility of the B-mode image is improved by changing the transparency of the information indicating the position of the blood vessel region to be higher, the problem in the gate setting is solved.

In the following description, the transmittance is one of color information set for image data or the like, and refers to a degree to which an image is transparent and information behind is transmitted. For example, it is possible to achieve an expression (display) in which the back is seen through a translucent image whose transmittance is set to a predetermined value. Further, the transparency includes the concept of color saturation. Provided that the transmittance and the color saturation are inversely related to each other, the color saturation decreases as the transmittance increases. For example, when the transmittance is 100, the color saturation is 0.

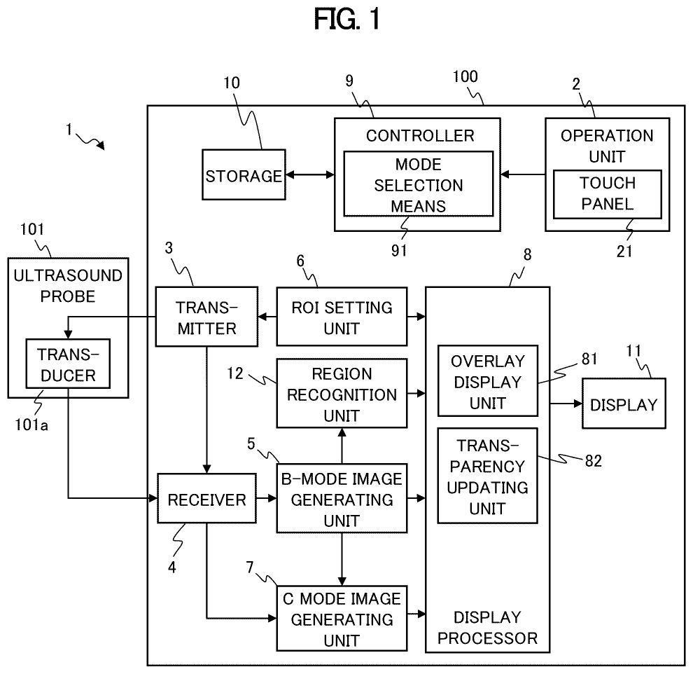

FIG. 1 is a block diagram illustrating the configuration of an ultrasound diagnostic apparatus 1 according to an embodiment. The ultrasound diagnostic apparatus 1 is configured such that an ultrasound probe 101 is connected to a main body 100 via a cable.

The main body 100 of the ultrasonic diagnostic apparatus 1 includes an operation unit 2, a transmitter 3, a receiver 4, a B-mode image generation unit 5, and an ROI (Region Of Interest) unit 6, a C-mode image generating unit 7, a display processor 8, a controller 9, a storage 10, a display 11, and a region recognition unit 12.

In the ultrasound probe 101, a plurality of transducers (piezoelectric transducer elements) 101a are arranged in one dimensional direction. Each of the transducers 101a is applied with a drive signal from the transmitter 3 to be described later to generate ultrasonic waves, and a plurality of ultrasonic waves interfere with each other to generate an ultrasonic beam. The ultrasonic beam is radiated to the subject. Next, the reflected ultrasonic waves (echoes) from the inside of the subject are received by the plurality of transducers 101a, converted into electrical signals, and output to the receiver 4 described later.

The operation unit 2 is a display instruction unit that detects an operation instruction of a user and notifies the controller 9 of a mode command based on the operation instruction of the user. For example, the operation unit 2 notifies the controller 9 of which of a mode for displaying a B-mode image and a mode for superimposing and displaying a C-mode image on a B-mode image is to be executed, based on an operation instruction from the user. The B-mode image is an ultrasonic image in which the amplitude of reflected ultrasonic waves is expressed by brightness (luminance), and the C-mode image is a color flow mode image.

The operation unit 2 also includes a function of receiving a designation input of the position of the ROI for displaying the C-mode image on the B-mode image by the user.

Here, the C-mode image includes a V-mode in which a flow velocity and a direction of the blood flow are displayed in color by a blood flow velocity V as a blood flow signal indicating a state of the blood flow, a P-mode in which power of the blood flow is displayed in color by power P of the blood flow as the blood flow signal, and a V-T mode in which the flow velocity and dispersion of the blood flow are displayed in color based on the blood flow velocity V and a dispersion T as the blood flow signal.

When detecting the user's operation instruction for C-mode image display, the operation unit 2 further detects the above-described display mode instruction. The display modes of the C-mode image display may include a T (dispersion) mode, a dP (directed power) mode, and the like. Thus, the display modes of the C-mode image display include the color Doppler mode (V mode, V-T mode, etc) and the power Doppler mode (P mode, etc).

Further, the operation unit 2 detects a user operation instruction of a puncture mode in which the puncture needle inserted with assistance of the puncture adapter or the guide is highlighted in the B-mode image, and notifies the controller 9 of the mode command.

In addition, the operation unit 2 detects a user operation instruction of a blood vessel display mode in which information indicating a position of a blood vessel region recognized from a B-mode image of an ultrasonic echo of a subject by using an AI model of machine learning or deep learning which has been learned is superimposed and displayed on a display screen of the B-mode image. Then, the operation unit 2 notifies the controller 9 of the blood vessel display mode command as a user operation instruction.

Further, the operation unit 2 detects a user operation instruction of a nerve display mode in which information indicating the position of the nerve region recognized from the B-mode image of the ultrasonic echo of the subject by using an AI model of machine learning or deep learning that has been learned is superimposed and displayed on the display screen of the B-mode image. Then, the operation unit 2 notifies the controller 9 of a nerve display mode command as a user operation instruction.

More specifically, the display processor 8 displays an instruction button on the display 11 which displays a B-mode image or the like to be described later. The operation unit 2 detects a tap operation of an instruction button by the touch panel 21 and sets the tap operation as an operation instruction of the user. Note that it is needless to say that the detection of the user's operation instruction is not limited to the touch panel 21 but may be performed by other detection means.

The transmitter 3 transmits a high-voltage drive signal at a predetermined timing to the transducer 101a of the ultrasonic probe 101 in order to irradiate the subject with an ultrasonic beam.

When a color Doppler image is displayed, the transmitter 3 performs transmission processing for the C mode for displaying a blood flow image in addition to transmission processing for displaying a B mode image under the control of the controller 9. At this time, the transmitter 3 performs, for example, the B-mode scanning and the C-mode scanning alternately.

In the C-mode scanning, while the ultrasonic beam is repeatedly transmitted n times in the same line direction, the ultrasonic beam is transmitted in all directions (all lines) of the ROI set by the ROI setting unit 6. For example, n is a dozen times such as 16, and the number of repetitions is referred to as the number of ensembles. Further, the transmitter 3 designates additional information of the transmission processing for the B-mode image or the transmission processing for the C-mode image at the time of the transmission processing, and outputs the additional information to the receiver 4.

The receiver 4 generates a digital reception signal by amplifying, A/D converting, and phasing and adding an analog electric signal converted based on the reflected ultrasonic wave (echo) received by the ultrasonic probe 101. Further, the receiver 4 acquires the additional information from the transmitter 3, and outputs the reception signal to the B-mode image generation unit 5 if the acquired additional information is additional information for a B-mode image. When the acquired additional information is additional information for a C-mode image, the reception signal is output to the C-mode image generating unit 7. Hereinafter, the reception signal for generating the B-mode image is referred to as a “B-mode reception signal”, and the reception signal for generating the C-mode image is referred to as a “C-mode reception signal”.

Under the control of the controller 9, the B-mode image generator 5 performs envelope line detection, logarithmic conversion, and the like on the B-mode reception signals input from the receiver 4, adjusts the dynamic range and the gain, and converts the luminance, thereby generating B-mode image data, and outputs the B-mode image data to the display processor 8.

The ROI setting unit 6 sets the region of interest (ROI) of the C-mode image in the transmitter 3 and the display processor 8 according to the designation information of the ROI input by the user via the operation unit 2 by the control of the controller 9.

The C-mode image generation unit 7 generates C-mode image data in accordance with the C-mode reception signal input from the receiver 4 under the control of the controller 9, and outputs the C-mode image data to the display processor 8.

The display processor 8 superimposes the C-mode image data and the information indicating the position of the blood vessel region on the B-mode image data, and displays the result on the display 11. The display processor 8 displays the B-mode image generated by the B-mode image generator 5 on the display 11 in the display mode of the B-mode image.

Furthermore, in the display mode of the C-mode image, the display processor 8 superimposes, in the overlay display 81, the C-mode image generated by the C-mode image generating unit 7 at the position of the selected ROI on the B-mode image generated by the B-mode image generating unit 5, and displays the superimposed image on the display 11.

The controller 9 sets the operation content of each processing unit of the main body 100 and controls the entire operation of the main body 100. In particular, the mode selection means 91 of the controller 9 controls the display processor 8 to display setting buttons for a display mode and an operation type on the display 11. For example, the setting button is a button for setting a display board of a B-mode image or a button for setting a mode of Doppler image display in which a blood flow signal is detected using a Doppler mode to generate and display a Doppler image. In addition, the setting buttons include a setting button for a puncture mode in which a puncture needle is highlighted in a B-mode image to assist puncturing and a setting button for a blood vessel region display mode in which information indicating the position of a blood vessel region is overlaid in a B-mode image. Furthermore, the setting buttons include a setting button for a nerve region display mode in which information indicating the position of a nerve region in a B-mode image is overlaid and displayed.

Then, the operation unit 2 detects a tap of the setting button displayed on the display 11 by the user through the touch panel 21 provided on the display 11, and notifies the mode selection unit 91 of a user operation instruction corresponding to the setting button. The mode selection unit 91 sets the operation content of each processing unit according to the notified user operation instruction such as the B-mode image display, the Doppler image display, the puncture mode, the blood vessel region display mode, or the nerve region display mode.

Although it has been described above that the operation unit 2 detects that the user has tapped a setting button for a display mode or an operation type displayed on the display 11, physical operation switches corresponding to operation content of the respective processing parts may be provided so that the operation unit 2 detects a user operation.

The storage 10 stores ultrasonic image data, a gain matrix, a frame rate, and the like. The ultrasonic image data includes B-mode image data, C-mode image data, composite image data, and the like.

The display 11 is a so-called monitor display such as an LCD (Liquid Crystal Display) or an EL (Electro Luminescence) display for displaying the display image data output from the display processor 8. A transparent touch panel 21 is superimposed on a display surface of the display 11 so that a tap operation of a setting button by a user can be detected. Instead of the tapping operation on the setting button by the user, a mouse click on a setting button or the like may be performed to provide the user's operation instruction.

The region recognition unit 12 is a processing unit that, when the blood vessel region display mode is set, recognizes (determines) a blood vessel region in the B-mode image generated by the B-mode image generating unit 5, using, for example, a learned model obtained by learning a convolutional neural network with teacher data. Furthermore, the region recognition unit 12 is a processing unit that, when the nerve region display mode is set, recognizes (determines) a nerve region in the B-mode image generated by the B-mode image generating unit 5, for example, by using a learned model obtained by learning a convolutional neural network with teacher data.

That is, the region recognition unit 12 includes a learned model for recognizing the blood vessel region and a learned model for recognizing the nerve region, and recognizes (determines) the blood vessel region and the nerve region in the B-mode image according to the operation instruction of the user.

The region recognition unit 12 outputs the blood vessel region and the nerve region recognized by itself to the display processor 8 as information indicating the position of the blood vessel region or the nerve region. The information indicating the positions of the blood vessel region and the nerve region is, for example, a contour figure of the region or a figure in which the inside of the contour is painted in a predetermined color (for example, red), and includes position information of the region in the B-mode image. The information indicating the position of the region may be information on the coordinate position of a pixel determined to be the blood vessel region in the B-mode image. The display processor 8 superimposes, on the B-mode image, the information indicating the position of the region output from the region recognition unit 12 by the overlay display unit 81, and displays the superimposed image on the display 11.

The transparency updating unit 82 is a processing unit that updates the transparency of the information displayed in a superimposed manner in real time when the information indicating the position of the blood vessel region recognized (determined) by the region recognition unit 12 and the information indicating the position of the nerve region are displayed in a superimposed manner on the B-mode image. For example, the transparency updating unit 82 updates the transparency of the translucent information to a high value, thereby improving the visibility of the superimposed B-mode image behind. The transparency updating unit 82 may be able to update the transparencies individually for the information indicating the position of the blood vessel region and the information indicating the position of the nerve region, or may update the transparencies in common for both.

Although details will be described later, the display processor 8 displays a spin button or a slider for setting the transparency on the display 11. The transparency updating unit 82 updates the transparency based on the operation information of the spin button or the slider of the user detected by the touch panel 21.

More specifically, the main unit 100 of the ultrasound diagnostic apparatus 1 according to the embodiment is realized by an information processing apparatus (e.g. a computer) that includes a central processing unit (CPU), a read only memory (ROM), a random access memory (RAM), and a large volume storage medium such as a HDD (Hard Disk Drive).

The CPU reads an ultrasound diagnosis program stored in the ROM or the HDD, develops the program in the RAM, and realizes the functions of the respective units of the main body 100 in accordance with the developed program. The RAM forms a work area for temporarily storing various programs to be executed by the CPU and data related to these programs. The ultrasonic diagnostic program may be stored in the form of a computer-readable program code and supplied to the ultrasonic diagnostic apparatus 1 by a recording medium, or may be transmitted via a network and supplied to the ultrasonic diagnostic apparatus 1.

FIG. 1 illustrates an example in which the ultrasonic diagnostic apparatus 1 of the embodiment is configured to include the main body 100 provided with the display 11 that displays a reflected ultrasonic wave (echo) and the ultrasonic probe 101. Not limited to this, it is possible to adopt a configuration in which each processing unit of the main body 100 is mounted on a server on a network as an ultrasonic diagnostic apparatus and the ultrasonic probe 101 and the display 11 are connected to the ultrasonic diagnostic apparatus via the network.

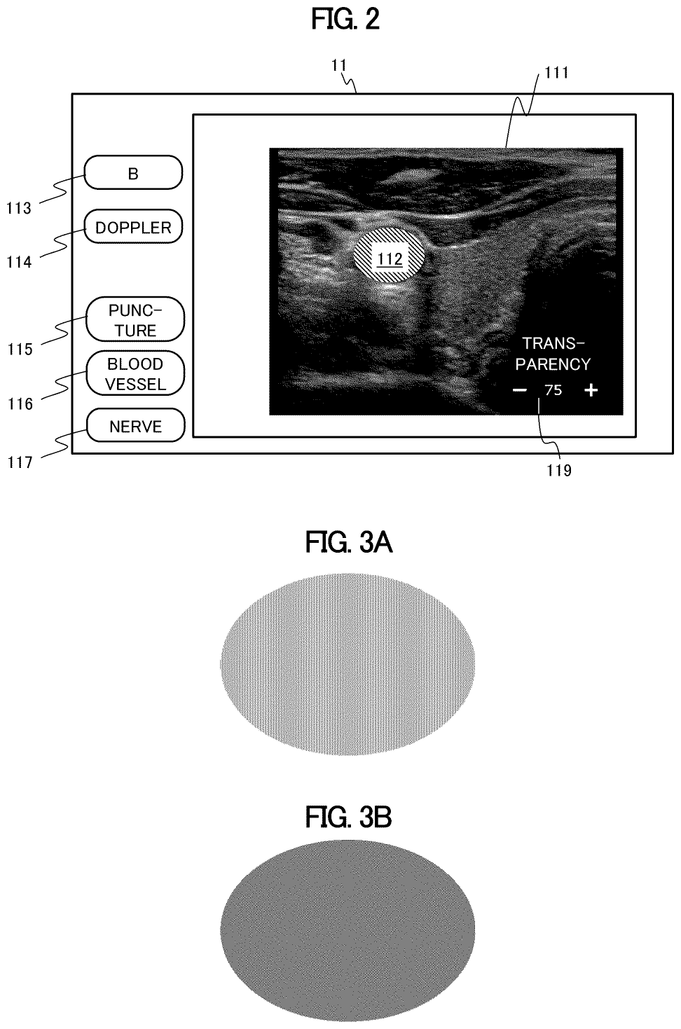

FIG. 2 is a view illustrating a display example of the display 11. On the display 11 in FIG. 2, blood vessel region information 112 indicating the position of the blood vessel region is superimposed and displayed on the B-mode image 111. The blood vessel region information 112 is a contour figure of the blood vessel region or a figure filled with a predetermined color (e.g., red) inside the contour. A setting button 119 for updating the transparency of the blood vessel region information 112 in real time is displayed on the display 11. That is, the display processor 8 superimposes the blood vessel region information 112 indicating the position of the blood vessel region on the B-mode image 111 and displays the superimposed image on the display 11.

Further, buttons for instructing and setting the operation of the ultrasonic diagnostic apparatus 1 by being tapped by the user are displayed on the display 11 as the operation unit 2. The B button 113 is a button for setting a display mode of a B-mode image. The Doppler button 114 is a button for setting a color Doppler display mode in which a color Doppler image is superimposed and displayed on a B-mode image.

Furthermore, a blood vessel button 116, a puncture button 115, and a nerve button 117 are displayed on the display 11. Note that FIG. 2 shows only buttons related to the operation of the ultrasonic diagnostic apparatus 1 of the embodiment described below, and omits the display of the other buttons.

The blood vessel button 116 is display instruction means for instructing a blood vessel display mode in which the blood vessel region information 112 recognized from the B-mode image is superimposed and displayed on the B-mode image. The puncture button 115 is a display instruction means for giving an instruction to display a puncture mode in which a puncture needle is highlighted. The nerve button 117 is display instruction means for instructing a nerve display mode in which nerve region information 118 (not illustrated) recognized from the B-mode image is superimposed and displayed on the B-mode image. That is, the display processor 8 displays the B button 113, the Doppler button 114, the blood vessel button 116, the puncture button 115, and the nerve button 117 on the display 11.

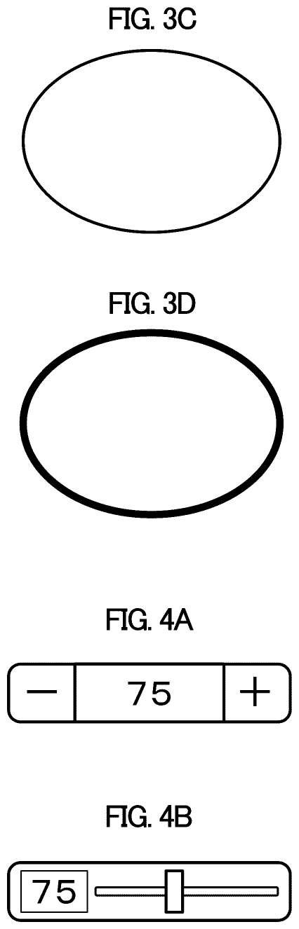

Next, the shapes of the blood vessel region information 112 and the neural region information 118 will be described with reference to FIGS. 3A to 3D. Hereinafter, the information indicating the position of the blood vessel region or the nerve region is referred to as information indicating a position. In the description, the information indicating the position is shown as an elliptical shape, but the elliptical shape is used only for illustration. The shape of the information indicating the position is an outline graphic of the area recognized (determined) by the region recognition unit 12 or a graphic including the area.

FIG. 3A illustrates a state in which the information indicating the position is an outline graphic or a graphic containing a region that is filled with a color having a predetermined transmittance.

In FIG. 3B, the information indicating the position is in a state in which an outline graphic or a graphic containing a region is filled with a color having a lower transparency than the transparency of FIG. 3A. That is, since the transmittance is lower than that in FIG. 3A, the white paper behind is illustrated in dark gray.

In FIG. 3C, the information indicating the position is an outline figure or a figure including a region and is in a state of an outline line indicated by a color having a predetermined transmittance, that is, in a state of color information in which a boundary region (peripheral part) of the recognized blood vessel region is recognizably displayed in a frame shape. At this time, the outline or the inside of the frame is made transparent so that the B-mode image behind can be seen. The boundary region of the blood vessel region refers to a peripheral portion, and the ultrasonic diagnostic apparatus 1 forms an annotation in a frame shape at this time.

FIG. 3D shows a state in which the line width of a contour line (frame line) in the information indicating the position in FIG. 3C is set in accordance with the transparency. In particular, as the transmittance increases, the line width of the outline (frame line) may be increased. Accordingly, even in a state where the transmittance is high, the contour line (frame line) can be easily visually recognized.

Next, with reference to FIGS. 4A and 4B, a configuration of a setting button for updating the transparency of the information indicating the position of the blood vessel region or the information indicating the position of the nerve region in real time will be described. The setting buttons for updating the transparency are provided on the display 11 individually for the information indicating the position of the blood vessel region and the information indicating the position of the nerve region, or are provided in common (for both).

FIG. 4A is a view illustrating a setting button for updating the transparency, which is formed by a spin button. In the spin button, a minus mark for decreasing a numerical value is arranged on the left, and a plus mark for increasing a numerical value is arranged on the right. When the user taps a minus mark, the transmittance decreases, and when the user taps a plus mark, the transmittance increases.

FIG. 4B is a diagram illustrating a setting button that is formed of a slider and updates transparency. The transparency is increased or decreased by a user operation of tapping and sliding the bar.

The transparency updating unit 82 sets the maximum value of the setting range of the transparency by the operation of the spin button or the slider to be equal to or smaller than a predetermined threshold value. Thus, the information indicating the position of the blood vessel region or the information indicating the position of the nerve region to be superimposed on the B-mode image is prevented from becoming completely transparent. This will prevent a recognized area from being overlooked.

When the transparency is set to be equal to or greater than a predetermined threshold value by a user operation and the diagnosis is ended, the transparency updating unit 82 resets the transparency to be equal to or less than the predetermined threshold value when the diagnosis is resumed and the blood vessel region display mode or the nerve region display mode is executed. That is, the transparency updating unit 82 sets the transparency to a predetermined initial value (a value equal to or smaller than a predetermined threshold value) in response to a start instruction such as the start of diagnosis or power-on without storing the transparency changed by the user operation of the setting button in response to an end instruction.

Thus, for example, when the emphasis display of the blood vessel region or the nerve region is performed again after the transmittance is changed to 100% and the process is completed, the emphasis display cannot be visually recognized if the transmittance is 100%, and thus there is a possibility that it is erroneously recognized that there is no blood vessel region or nerve region, but it is possible to suppress the erroneous recognition by resetting the transmittance to be equal to or smaller than the predetermined threshold value.

When the display of the information indicating the position of the blood vessel region or the nerve region after the change of the transmittance is started, the transmittance updater 82 may reset the transmittance to a value equal to or smaller than a predetermined threshold and may change the transmittance to a predetermined color tone.

Further, the transparency updating unit 82 may include a notification unit that notifies that the transparency is changed to exceed a predetermined value when the transparency is set to be equal to or greater than a predetermined threshold value by a user operation of the setting button. Thus, the user can recognize that the information indicating the position of the blood vessel region or the nerve region is set beyond the certain transmission state. A case is considered in which the transparency is set to 100% when the predetermined emphasis display is performed again. In this case, even if the blood vessel emphasis button or the muscle emphasis button is pressed, the blood vessel or the muscle might not be displayed and the next operator might determine that there is no blood vessel or muscle. This notification prevents this from happening. Further, the ultrasound diagnostic apparatus 1 may be activated in a state in which a sufficient color is always applied at the time of activation. Thus, a situation in which the next operator determines that there is no blood vessel or muscle can be prevented.

The notification unit displays, for example, a pop-up window stating that the transmittance has been changed beyond a predetermined value. Furthermore, the setting button may be highlighted, for example, inverted, or blinked to provide a warning that the transmittance has exceeded a predetermined value.

Next, a description will be provided on an operation of superimposing and displaying the information indicating the position of the blood vessel region on the B-mode image as illustrated in FIG. 2 by the user's tap on the blood vessel button 116 when the ultrasonic diagnostic apparatus 1 displays the B-mode image on the display 11 based on the user's tap on the B button 113 (see FIG. 2).

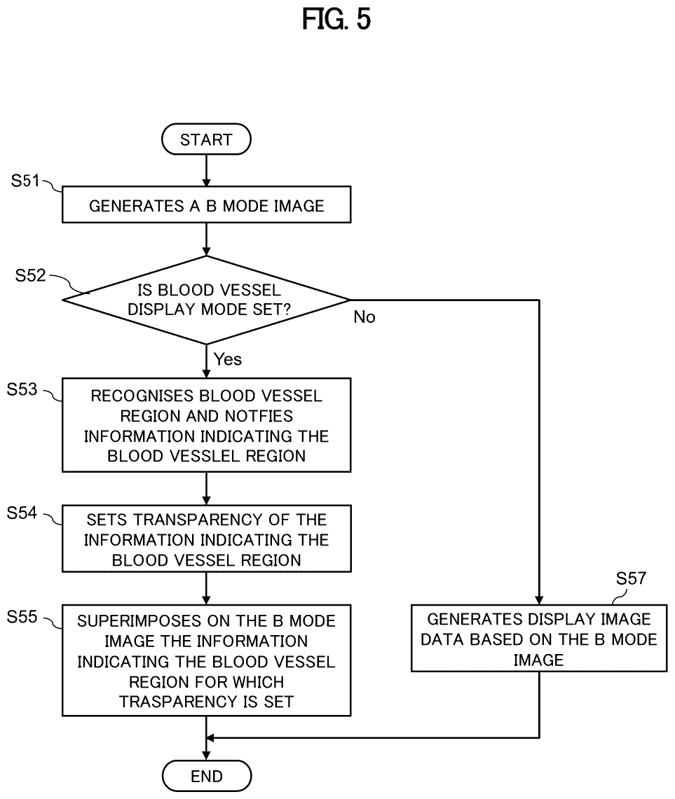

FIG. 5 is a flowchart illustrating processing for one frame of a reflected ultrasonic wave (echo) in the ultrasonic diagnostic apparatus 1. The controller 9 of the ultrasonic diagnostic apparatus 1 repeats the processing of FIG. 5 at a predetermined cycle.

In step S51, the B-mode image generating unit 5 generates a B-mode image based on the reflected ultrasonic waves.

In step S52, the display processor 8 determines, based on detection of a tap on the blood vessel button 116 indicating the blood vessel display mode on the touch panel 21 of the operation unit 2, whether or not the mode is the blood vessel display mode. Then, if the mode is the blood vessel display mode (yes in step S52), the process proceeds to step S53. If the mode is not the blood vessel display mode (no in step S52), the process proceeds to step S57.

In step S53, the region recognition unit 12 recognizes (determines) a blood vessel region by the learned model in the B-mode image generated in step S51, and notifies the display processor 8 of the recognized blood vessel region as information indicating the position of the blood vessel region.

In step S54, the operation unit 2 detects presence or absence of a user's tap operation on the setting button 119 for the transparency of the blood vessel region on the touch panel 21. When the user performs a tap operation, the transparency updating unit 82 of the display processor 8 sets (updates) the transparency of the information indicating the position of the blood vessel region in accordance with the user's tap operation.

In step S55, the overlay display 81 of the display processor 8 superimposes the B-mode image and the information indicating the position of the blood vessel region in which the transparency set (updated) by the transparency updating unit 82 is reflected, displays the superimposed image on the display 11, and ends the process.

In step S57, the display processor 8 displays the B-mode image generated in step S51 on the display 11, and ends the processing. The blood vessel region information 112 is not superimposed on the B-mode image generated in step S51.

As described above, the ultrasonic diagnostic apparatus 1 detects the setting of the transmittance for each frame of the B-mode image, thereby displaying the information indicating the position of the blood vessel region in a superimposed manner in real time and changing the transmittance of the information indicating the position of the blood vessel region. Thus, a decrease in the visibility of the B-mode image transmitted through the blood vessel region can be suppressed, and the state behind the blood vessel region can be grasped.

Although the flowchart of FIG. 5 illustrates that the setting of the transmittance is detected for each frame of the B-mode image, the processing of the setting of the transmittance in Step S54 may be thinned out within a range in which a change in the transmittance of the information indicating the position of the blood vessel region is allowable.

As in the flowchart of FIG. 5, when the ultrasonic diagnostic apparatus 1 superimposes and displays only the information indicating the position of the blood vessel region on the B-mode image, the region recognition unit 12 does not needs to have a learned model for recognizing the nerve region. In addition, it is not necessary to display the nerve button 117 illustrated in FIG. 2 on the display 11, and it is not necessary for the operation unit 2 to detect the user operation of the nerve button 117.

Conversely, the ultrasonic diagnostic apparatus 1 may superimpose and display only the information indicating the position of the nerve region on the B-mode image instead of the information indicating the position of the blood vessel region. In this case, the ultrasonic diagnostic apparatus 1 does not need a learned model for recognizing a blood vessel region and processing related to the blood vessel button 116.

Next, an example will be described in which the ultrasound diagnostic apparatus 1 superimposes and displays information indicating the position of a blood vessel region and information indicating the position of a nerve region on a B-mode image, and the transparency of the information indicating the position of the blood vessel region and the information indicating the position of the nerve region can be changed.

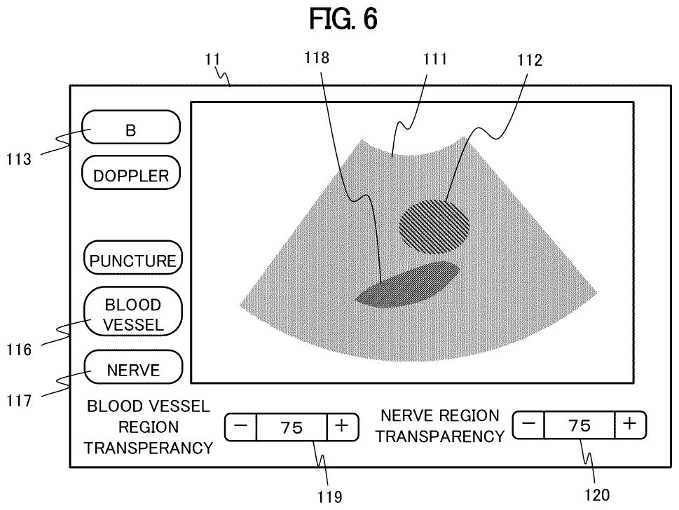

FIG. 6 shows the display 11 on which the ultrasonic diagnostic apparatus 1 displays the B-mode image 111 by the tap operation of the B button 113 by the user, then detects the tap operations of the blood vessel button 116 and the nerve button 117 by the user, and superimposes and displays the blood vessel region information 112 and the nerve region information 118 indicating the position of the nerve region on the B-mode image 111.

The display 11 is provided with a blood vessel region transmittance setting button 119 and a nerve region transmittance setting button 120, and the transmittances of the blood vessel region information 112 and the nerve region information 118 can be changed in real time in accordance with a user operation.

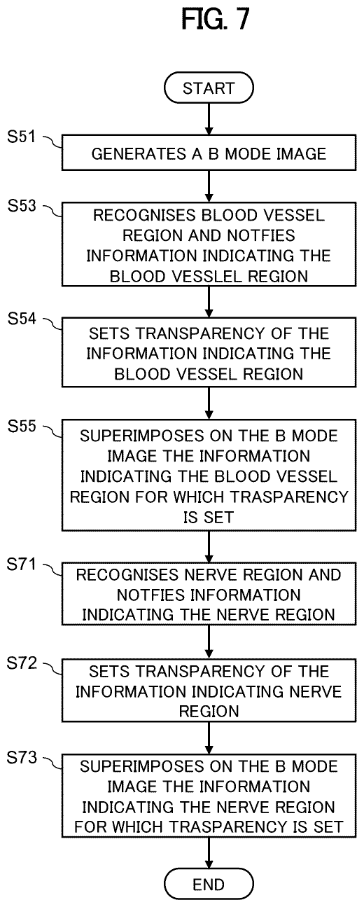

FIG. 7 is a flowchart illustrating an operation of superimposing and displaying information indicating the positions of a blood vessel region and a nerve region on a B-mode image at the same time.

First, in step S51 and steps S53 to S55, the display processor 8 superimposes the B-mode image and the blood vessel region information 112 indicating the position of the blood vessel region in which the transparency is reflected, and displays the superimposed image on the display 11. Details of each step are the same as those in FIG. 5, and thus the description thereof is omitted here.

In step S71, the region recognition unit 12 recognizes (determines) a neural region in the B-mode image generated in step S51 using the learned model, and notifies the display processor 8 of the recognized neural region as the neural region information 118 indicating the position of the neural region.

In step S72, the operation unit 2 detects the presence or absence of a tap operation performed by the user on the transparency setting button 120 on the touch panel 21. Then, when the user performs a tap operation, the transparency updating unit 82 of the display processor 8 sets (updates) the transparency of the nerve region information 118 according to the user's tap operation.

In step S73, the overlay display unit 81 of the display processor 8 superimposes and displays, on the display 11, information indicating the position of the neural area reflecting the transparency set in step S72, and ends the processing.

As described above, the ultrasonic diagnostic apparatus 1 superimposes and displays the information indicating the positions of the blood vessel region and the nerve region on the B-mode image at the same time, but the information indicating the position of the blood vessel region and the information indicating the position of the nerve region may overlap each other. In this case, even if the transmittance is adjusted, the visibility of the B-mode image behind may not be improved.

When the pieces of information indicating the positions of the blood vessel region and the nerve region overlap each other as described above, one of the information indicating the position of the blood vessel region and the information indicating the position of the nerve region may be preferentially displayed.

The preferential display means that, in a case where a plurality of pieces of information indicating the position of the tissue region recognized using the learned model are present, when the plurality of pieces of information recognize the same region, only one of the pieces of information is displayed. For example, in a case where information indicating the position of a blood vessel region and information indicating the position of a nerve region are present, when the ultrasonic diagnostic apparatus 1 recognizes the same region on an image as the blood vessel region information and the nerve region information, the region is either the blood vessel region information or the nerve region information.

In other words, only one of them is displayed.

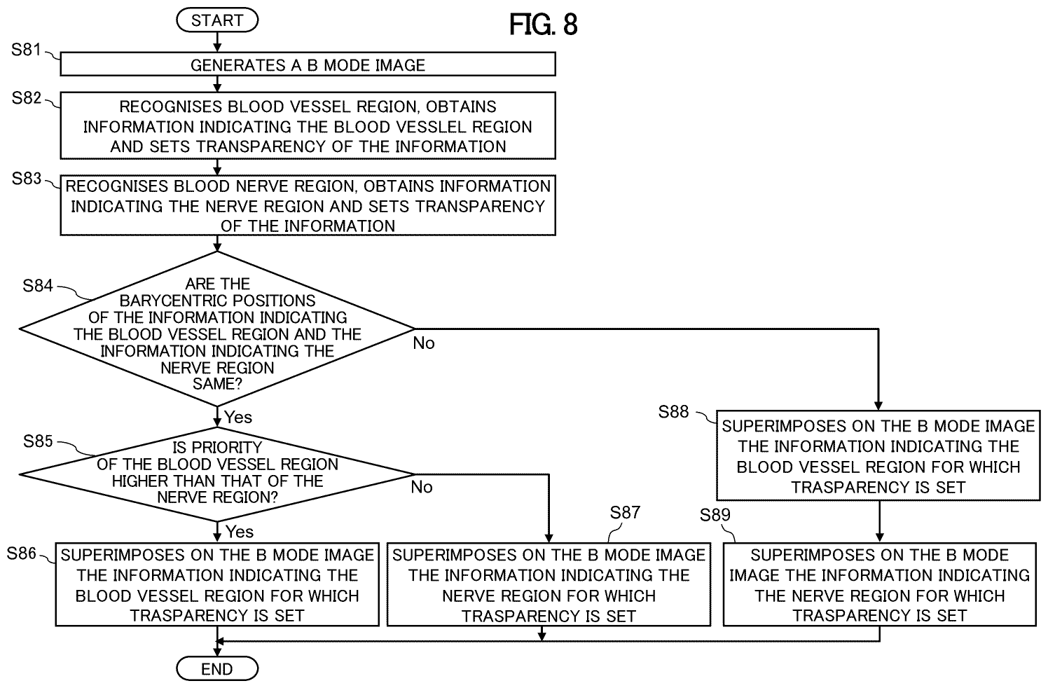

FIG. 8 is a flowchart illustrating an operation of preferentially displaying one of the information indicating the positions of the blood vessel region and the nerve region.

In step S81, the B-mode image generator 5 generates a B-mode image based on the reflected ultrasonic waves (echoes).

In step S82, the display processor 8 recognizes a blood vessel region, obtains information indicating the position of the blood vessel region, and sets the degree of transparency. Specifically, the region recognition unit 12 recognizes (determines) a blood vessel region in the B-mode image generated in step S81 using the learned model, and notifies the display processor 8 of the recognized blood vessel region as information indicating the position of the blood vessel region. Further, the operation unit 2 detects the presence or absence of a tap operation by the user on the setting button 119 for the transmittance of the blood vessel region on the touch panel 21. When the user performs a tap operation, the transparency updating unit 82 of the display processor 8 sets (updates) the transparency of the information indicating the position of the blood vessel region in accordance with the user's tap operation.

In step S83, the display processor 8 recognizes a nerve region, obtains information indicating the position of the nerve region, and sets the degree of transparency. Specifically, the region recognition unit 12 recognizes (determines) the neural region by the learned model in the B-mode image generated in step S81, and notifies the display processor 8 of the recognized neural region as information indicating the position of the neural region. Further, the operation unit 2 detects the presence or absence of a tap operation by the user on the setting button 120 for the transmittance of the nerve region on the touch panel 21. When the user performs a tap operation, the transparency updating unit 82 of the display processor 8 sets (updates) the transparency of the information indicating the position of the nerve region in accordance with the user's tap operation.

In step S84, the display processor 8 determines whether the barycentric positions of the information indicating the position of the blood vessel region and the information indicating the position of the nerve region are the same by determining whether the difference between the barycentric positions is equal to or smaller than a predetermined value. When the difference between the barycentric positions is equal to or smaller than the predetermined value, it is determined that the barycentric positions are the same (Yes), and the process proceeds to step S85. When the difference between the barycentric positions is larger than the predetermined value, it is determined that the barycentric positions are not the same (No), and the process proceeds to step S88.

The display processor 8 may determine the presence or absence of the overlap depending on whether or not the size of the area of the overlap between the information indicating the position of the blood vessel region and the information indicating the position of the nerve region is equal to or larger than a predetermined value as the determination condition in step S84.

In step S85, the display processor 8 determines whether or not the priority of the information indicating the positions of the blood vessel region is higher than the priority of the information indicating the positions of the nerve region. If the priority of the blood vessel region is higher than the priority of the nerve region (YES in step S85), the process proceeds to step S86. If the priority of the blood vessel region is lower than or equal to the priority of the nerve region (NO in step S85), the process proceeds to step S87.

In step S86, the overlay display unit 81 of the display processor 8 superimposes and displays, on the display 11, the B-mode image and information indicating the position of the blood vessel region in which the transparency set (updated) by the transparency updating part 82 is reflected, and ends the processing of FIG. 8.

In step S87, the overlay display unit 81 of the display processor 8 superimposes and displays the B-mode image and the information indicating the position of the neural region reflecting the transparency set (updated) by the transparency updating unit 82 on the display 11, and the process of FIG. 8 ends.

In step S88, the overlay display unit 81 of the display processor 8 superimposes and displays the B-mode image and the information indicating the position of the blood vessel region reflecting the transparency set (updated) by the transparency updating unit 82 on the display 11, and the process proceeds to step S89.

In step S89, the overlay display unit 81 of the display processor 8 superimposes and additionally displays, on the display 11, information indicating the position of the neural area in which the transparency set in step S83 is reflected, and ends the processing in FIG. 8.

In the above description, it has been described that the region recognition unit 12 includes each of the learned models for recognizing the blood vessel region and the nerve region, and recognizes the blood vessel region and the nerve region in the B-mode image according to the operation instruction of the blood vessel region display mode or the nerve region display mode from the user.

In addition, the region recognition unit 12 includes a learned model for recognizing a muscle tissue region, and recognizes a blood vessel region, a nerve region, and a muscle tissue region in a B-mode image. Furthermore, the display 11 is provided with a muscle tissue button which is display instruction means for instructing a muscle tissue display mode in which information indicating the position of the muscle tissue region is superimposed and displayed. Then, the display processor 8 may superimpose and display at least one of the information indicating the position of the blood vessel region, the information indicating the position of the nerve region, and the information indicating the position of the muscle tissue region on the B-mode image.

Further, a setting button capable of changing the transparency of the information indicating the position of the muscle tissue region is provided, and the transparency updating unit 82 sets the transparency of the information indicating the position of the muscle tissue region according to the setting of the setting button. Then, the overlay display unit 81 may superimpose the B-mode image and the information indicating the position of the muscle tissue region reflecting the transmittance.

Next, the relation between color Doppler display that operates in response to a tap of the Doppler button 114 (see FIG. 2 and FIG. 6) and the function of changing the transmittance of information indicating the position of a blood vessel region will be described.

The ultrasound diagnostic apparatus 1 superimposes and displays, on the B-mode image 111, the blood vessel region information 112 whose transparency can be changed by the overlay display unit 81 in response to a user's tap on the blood vessel button 116. Thereafter, the ultrasound diagnostic apparatus 1 causes the overlay display unit 81 to display the C-mode image superimposed on the B-mode image 111 in response to the user's tap operation on the Doppler button 114. This display mode is referred to as color Doppler display.

That is, while the information indicating the position of the blood vessel region is displayed by the live scanning in the B mode, the ultrasonic diagnostic apparatus 1 can enable the color Doppler display, and displays the information indicating the position of the blood vessel region and the C mode image in a superimposed manner at the same time. Thus, even if the information indicating the position of the blood vessel region and the color Doppler display overlap each other, the user can easily visually recognize the color Doppler display by changing the transparency of the blood vessel region information 112 to be higher.

Furthermore, the ultrasound diagnostic apparatus 1 may be capable of ending the color Doppler display while the live scanning is being performed in the B mode and the information indicating the position of the blood vessel region is being displayed.

Here, a description will be provided on the display when the ultrasound diagnostic apparatus 1 detects a tap of the puncture button 115 (see FIG. 2) instructing to display the puncture mode in which a puncture needle is highlighted, and displays the puncture mode.

The ultrasound diagnostic apparatus 1 is configured such that the transparency of the blood vessel region information 112 or the nerve region information 118 superimposed and displayed on the B-mode image in the puncture mode can be changed by a user operation. Thus it is possible to keep the visibility of the B-mode image behind the blood vessel region information 112 or the nerve region information 118. Therefore, the user can easily grasp the blood vessel region or the nerve region, and also visually recognize the puncture needle on the B-mode image, thus allowing the puncture procedure to be favorably performed.

When the information indicating the position of the blood vessel region recognized using the learned model is superimposed on the B-mode image and highlighted, there is a problem of recognition accuracy of the blood vessel region, and the portion of the blood vessel and the region in the vicinity thereof may be highlighted. However, there might be actually nerves near the blood vessel. That is, there is a possibility that a nerve exists in a region in the vicinity of the blood vessel.

In the case of opaque highlighting, the nerve may be hidden and invisible, but in the ultrasound diagnostic apparatus 1 described above, the information indicating the position of the blood vessel region is made translucent and highlighted so that the nerve is not hidden and invisible.

In addition, in a case of opaque highlighted display, a region of a tissue in the B-mode image is hidden and becomes invisible, and a boundary of a recognized tissue becomes invisible, and therefore, it is not clear whether the tissue is really correctly recognized. However, since the translucent emphasis display is performed in the ultrasonic diagnostic apparatus 1, the boundaries of the tissues in the B-mode image can be visually recognized, and thus it is possible to know that the tissues are correctly recognized.

According to the present embodiment, when the information indicating the position of the blood vessel region is superimposed on the B-mode image and is displayed in real time, the transparency of the information indicating the position of the blood vessel region may be changed and displayed in real time by the operation of changing the transparency by the operation unit 2. When the ultrasonic diagnostic apparatus 1 displays a B-mode image in real time while performing live scanning in the B-mode, the ultrasonic diagnostic apparatus 1 can perform live annotation display of a blood vessel by artificial intelligence. The ultrasonic diagnostic apparatus 1 can change the transparency in the state of the live display while the overlay of the annotation display is displayed live and the blood vessel is highlighted live.

Furthermore, the present invention is not limited to the above-described examples and includes various modification examples. The above-described embodiments have been described in detail for easy understanding of the present invention, and the present invention is not necessarily limited to those having all the configurations described above. Furthermore, part of the configuration of an embodiment can be replaced with the configuration of another embodiment, and the configuration of another embodiment can be added to the configuration of an embodiment.

Although embodiments of the present invention have been described and illustrated in detail, the disclosed embodiments are made for purposes of illustration and example only and not limitation. The scope of the present invention should be interpreted by terms of the appended claims.

### DESCRIPTION OF REFERENCE NUMERALS

1 ULTRASONIC DIAGNOSTIC APPARATUS
2 OPERATION UNIT (OPERATION MEANS)
3 TRANSMITTER
4 RECEIVER
5 B-MODE IMAGE GENERATING UNIT
6 ROI SETTING UNIT
7 C MODE IMAGE GENERATING UNIT
8 DISPLAY PROCESSOR
81 OVERLAY DISPLAY UNIT
82 TRANSPARENCY UPDATING UNIT
9 CONTROLLER
91 MODE SELECTION MEANS
10 STORAGE
11 DISPLAY
12 REGION RECOGNITION UNIT
100 BODY
101 ULTRASOUND PROBE

What is claimed is:

1. An ultrasound diagnostic apparatus, comprising
a display on which information indicating a position of a blood vessel region recognized using a learned model is displayed; and
an operation unit which is adapted to change a degree of transparency of the information indicating the position of the blood vessel region.

2. The ultrasound diagnostic apparatus according to claim 1, wherein the operation unit is provided on a screen of the display.

3. The ultrasound diagnostic apparatus according to claim 1, wherein the information indicating the position of the blood vessel region is semitransparent color information to be superimposed on an ultrasound image.

4. The ultrasound diagnostic apparatus according to claim 1, wherein the degree of transparency is changeable by the operation unit during real-time display of a B-mode image on which the information indicating the position of the blood vessel region is superimposed.

5. The ultrasound diagnostic apparatus according to claim 1, wherein the information indicating the position of the blood vessel region is color information indicating that the blood vessel region recognized using the learned model is painted in a predetermined color, or the information indicating the position of the blood vessel region is color information in which a boundary region of the blood vessel region recognized using the learned model is displayed in a frame shape.

6. The ultrasound diagnostic apparatus according to claim 1, wherein the display shows information indicating a position of a nerve region recognized using a learned model, and the ultrasound diagnostic apparatus further includes an operation unit that is adapted to change a degree of transparency of the information indicating the position of the nerve region.

7. The ultrasound diagnostic apparatus according to claim 1, wherein the display shows information indicating a position of a nerve region recognized using a learned model and the information indicating the position of the blood vessel region at the same time, and wherein
the degree of transparency of the information indicating the position of the blood vessel region and a degree of transparency of the information indicating the position of the nerve region are independently changeable.

8. The ultrasound diagnostic apparatus according to claim 7, wherein the display shows the information indicating the position of the nerve region recognized using the learned model and the information indicating the position of the blood vessel region at the same time, and wherein
the ultrasound diagnostic apparatus further includes a first operation unit which is adapted to change a degree of transparency of the information indicating the position of the blood vessel region; and a second operation unit which is adapted to change a degree of transparency of the information indicating the position of the nerve region.

9. The ultrasound diagnostic apparatus according to claim 1, wherein the display shows information indicating a position of a nerve region recognized using a learned model and the information indicating the position of the blood vessel region at the same time, and wherein
the ultrasound diagnostic apparatus further includes one operation unit adapted to change the degree of transparency of the information indicating the position of the blood vessel region and a degree of transparency of the information indicating the position of the nerve region.

10. The ultrasonic diagnostic apparatus according to claim 6, wherein one of the information indicating the position of the blood vessel region and the information indicating the position of the nerve region is preferentially displayed.

11. The ultrasound diagnostic apparatus according to claim 1, wherein the display is capable of displaying information indicating a position of a muscle tissue region recognized by a learned model, information indicating a position of a nerve region recognized by a learned model, and the information indicating the position of the blood vessel region are displayable on the display at the same time.

12. The ultrasound diagnostic apparatus according to claim 11, wherein a degree of transparency of the information indicating the position of the muscle tissue region is changeable.

13. The ultrasound diagnostic apparatus according to claim 1, further including a controller configured to, in a case where the degree of transparency is set to be equal to or higher than a predetermined threshold and an end instruction is given, detect the end instruction and perform control to change the transparency to be equal to or lower than the predetermined threshold.

14. The ultrasound diagnostic apparatus according to claim 1, further including a notification unit that provides a notification when the degree of transparency is set to be equal to or higher than a predetermined threshold.

15. The ultrasound diagnostic apparatus according to claim 1, further including a setting unit adapted to set a maximum value of a setting range of the degree of transparency to be equal to or smaller than a predetermined threshold.

16. The ultrasound diagnostic apparatus according to claim 1, wherein a degree of transparency of color of the information indicating the position of the blood vessel region is set to be equal to or less than a predetermined threshold at the time of display activation for starting to display the information indicating the position of the blood vessel region.

17. The ultrasound diagnostic apparatus according to claim 1, wherein the ultrasound diagnostic apparatus is adapted to enable color Doppler display to display an overlay on the information indicating the position of the blood vessel region when a live scanning is performed in a B mode and the information indicating the position of the blood vessel region is displayed.

18. The ultrasound diagnostic apparatus according to claim 17, wherein the color Doppler display can be ended while the information indicating the position of the blood vessel region is being displayed by the live scanning in the B mode.

19. A non transitory storage medium which stores a program implemented by a computer, the program causing the computer to execute:
changing a degree of transparency of information indicating a position of a blood vessel region which is recognized using a learned model; and
displaying the information indicating the position of the blood vessel region.

20. An ultrasonic diagnostic method implemented by a computer, the method comprising causing the computer to execute:
changing a degree of transparency of information indicating a position of a blood vessel region which is recognized using a learned model, and;
displaying the information indicating the position of the blood vessel region.
